(19) United States
(12) Patent Application Publication
(10) Pub. No.: US 2025/0197445 A1
(43) Pub. Date: Jun. 19, 2025

(54) PREPARATION METHODS FOR A HIGHLY CONCENTRATED PD1 ANTIBODY SOLUTION BY ULTRAFILTRATION/DIAFILTRATION (UF/DF)

(71) Applicant: BeiGene Switzerland GmbH, Basel (CH)

(72) Inventors: Yu JI, Suzhou (CN); Xuening LI, Suzhou (CN); Jiankun DING, Guangzhou (CN); Bo QIU, Guangzhou (CN)

(73) Assignee: BeiGene Switzerland GmbH, Basel (CH)

(21) Appl. No.: 19/012,602

(22) Filed: Jan. 7, 2025

Related U.S. Application Data
(63) Continuation of application No. PCT/CN2023/106294, filed on Jul. 7, 2023.

(30) Foreign Application Priority Data
Jul. 12, 2022 (WO) PCT/CN2022/105070
Feb. 8, 2023 (WO) PCT/CN2023/074867

(51) Int. Cl. C07K 1/34; B01D 61/14; B01D 69/02; C07K 16/28

(52) U.S. Cl. CPC C07K 1/34; B01D 61/146; B01D 69/02; C07K 16/2818; B01D 2315/16; B01D 2317/02; B01D 2325/34

(57) ABSTRACT

The present disclosure provides for a preparation method of highly concentrated antibody solution that binds to human programmed death receptor 1 (PD1). This process is able to manufacture the high concentrated antibody solution by an ultrafiltration/diafiltration (UF/DF) unit operation described herein. The UF/DF preparation method comprises mainly a first ultrafiltration concentration step, a buffer solution diafiltration step, and a second ultrafiltration concentration step. The process has a broad operation parameter range and maintains antibody stability and integrity when compared to low concentration antibody preparation.

### CROSS-REFERENCE TO RELATED APPLICATIONS

This application is a continuation of International Patent Application No. PCT/CN2023/106294, filed Jul. 7, 2023, which claims priority from International Patent Application No. PCT/CN2022/105070, filed Jul. 12, 2022, and International Patent Application No. PCT/CN2023/074867, filed Feb. 8, 2023. The contents of these applications are incorporated herein by reference in their entirety.

### SEQUENCE LISTING

The instant application contains a sequence listing which has been submitted electronically in xml format, and is hereby incorporated by reference in its entirety. Said xml file was created on Jan. 7, 2025, is 16 bytes in size, and named 138881_1063_Sequence_Listing.xml.

### FIELD OF THE DISCLOSURE

The present disclosure is directed to methods of preparing a highly concentrated solution comprising antibodies or antigen binding fragments thereof that bind to human programmed death receptor 1 (PD1). This methodology is intended to manufacture a high concentrated antibody solution by ultrafiltration/diafiltration (UF/DF) as described herein. High concentration antibody solutions are useful, for example, in subcutaneous administration. The UF/DF preparation method comprises a first ultrafiltration concentration step, a buffer solution diafiltration step and a second ultrafiltration concentration step. This process has a broad operating parameter range and demonstrates that the high concentration solution prepared by this method preserves the antibody quality characteristics found in low concentration formulations.

### BACKGROUND OF THE DISCLOSURE

With the rapid development of antibody therapeutics, more and more are turning to subcutaneous formulations as opposed to intravenous (IV) formulation and administration, in order to reduce the clinical cost and improve the compliance of patients. For the subcutaneous route of administration of monoclonal antibody injections, the dose administered is usually in the range of 50 mg to 800 mg, while the maximum subcutaneous volume is generally limited to about 2 ml, which provides for a nominal volume to be delivered in a short period of time. Therefore, highly concentrated protein preparations require additional processes to obtain protein concentrations of up to 100 mg/ml or more without detriment to the antibody itself. The high concentration of monoclonal antibody presents many challenges for manufacturing processes, process scaling, and ultimately patient administration. One of the most significant challenges is high viscosity. Due to the properties of antibodies at high concentrations, therapeutic antibody formulations may form overly viscous solutions. In some instances, the ultrafiltration process can result in protein precipitation that occludes the membrane, resulting in either product loss or failure of the process. Another difficulty in concentrating high concentration monoclonal antibody solutions by ultrafiltration is that antibodies can aggregate to form masses and/or precipitates after the concentration process is finished. Lastly, if the final high concentration protein solution is obtained by modifying the filtration process, the concentrated antibody formulation needs to have a suitable viscosity for use in a disposable sterile syringe or a prefilled needle for subcutaneous administration. In the manufacturing process, ultrafiltration/diafiltration (UF/DF) is typically the final process to obtain the antibody concentration in the range of 10-60 mg/ml. However, the antibody dose for intravenous infusion is about one hundred milligrams to about one gram. In order to achieve the same pharmacokinetics and efficacy in subcutaneous administration by injecting an antibody solution under the skin, the ideal target antibody concentration during UF/DF can be as high as 150 mg/ml or above.

This high concentration creates technical challenges in the manufacturing process. First, highly concentrated antibody solutions can have high viscosity, which shows different hydrodynamic behavior in UF/DF. The mass transfer can be limited due to higher pressure on the membrane, resulting in decreased flux through the membrane, and can lead to membrane fouling. Secondly, there is a great difference between the initial feed protein concentration and the protein concentration in the final solution, during which 40 times concentration can be required. The volume change is also quite large, especially in commercial scale manufacturing. These factors play a role in the design of the UF/DF process and selection of skid. The UF/DF process setup should be able to handle large volume solution under high flowrate, and then be able to handle extreme low volume (10 or 20 times less) under relatively low flowrate for highly concentrated solution in the later processing phase. The range of pump and sensors, tubing diameter, flowmeter and dead volume cannot meet both the process requirements in the early phase and the later phase by using the conventional UF/DF process setup. Lastly, the characteristics of the primary amino acid sequence of the antibody is one of the major determinants of the properties of antibody solubility and/or stability in different formulations. The highly concentrated antibody solution can be modified in the final formulation, with low viscosity and high stability, by formulation and viscosity reducer screening and other stability studies. However, the antibody may not maintain its structural stability in the feed buffer solution with increasing antibody concentration in the first ultrafiltration step. Even if the antibody is stable in the feed buffer solution at concentrations of 150 mg/ml or above, the following diafiltration process can be extremely time consuming due to the high viscosity of the antibody solution without the addition of viscosity reducing chemicals, such as salts, amino acids, sugars, polyols, and surfactants, among others. The present disclosure provides for a novel preparation method of manufacturing a highly concentrated antibody solution by UF/DF steps.

### SUMMARY

The present disclosure provides a preparation method of a highly concentrated anti-human PD1 monoclonal antibody solution for subcutaneous administration by UF/DF unit operation, preferably Tislelizumab. The UF/DF process comprises the steps of:
1. Loading the feed material plus buffer into the UF/DF system with a starting protein concentration after viral filtration;
2. Ultrafiltrating the solution to obtain UF1 pool with an intermediate concentration in the UF1 step;
3. Diafiltrating the UF1 pool with DF buffer into final drug substance formulation buffer, preferably His-His HCl buffer, to obtain the DF pool;
4. Ultrafiltrating the DF pool into a high concentration protein solution as over concentrated pool with a required concentration;
5. Preparing the UF2 pool by combining the over concentrated pool with or without system flush, and diluting to the final high concentration formulation solution by adding surfactant, sugar stock solution to achieve the final drug substance target concentration.

In some embodiments, the feed material is in 50 mM acetate buffer, with different initial concentration from 3 g/L to 18 g/L. In some embodiments, the UF/DF membrane can be Pellion3 Ultracel™ 30 kDa, D membrane, Sartocon Slice ECO Hydrosart™ 30 kDa membrane or other 30 kDa or 50 kDa membrane. The membrane area can be adjusted according to the total protein amount for processing. In some embodiments, the membrane loading capacity is about 229.0 g/m², 585.2 g/m², 601.7 g/m², 739.7 g/m², preferably between 100 g/m² and 800 g/m².

In some embodiments, the transmembrane pressure (TMP) for UF1 is in 6-29 Psi range, preferably ˜14.5 Psi. The feed flowrate can be 4 L/min/m², 5 L/min/m² or up to 6 L/min/m². The protein concentration of the UF1 pool after the first ultrafiltration step can be 25 g/L, 50 g/L, 75 g/L. In some embodiments, UF1 pool concentration is 30 g/L, 70 g/L or any value between 25-75 g/L. In some embodiments, the VCF is from 3.41 to 10.23, preferably not more than 25 in UF1 step.

In some embodiments, the TMP for DF is in 6-29 Psi range, preferably ˜14.5 Psi. The feed flowrate can be 4 L/min/m², 5 L/min/m² or up to 6 L/min/m². The starting protein concentration of UF1 pool for DF step is within 25-75 g/L, preferably 50 g/L. The exchange volume number in DF step should be larger than 4, preferably 6or more.

In some embodiments, the TMP for UF2 is in 6-29 Psi range, preferably ˜14.5 Psi. In some embodiments, the feed flowrate can be about 0.5 L/min/m², 1 L/min/m², 2.5 L/min/m², 5 L/min/m² or up to 6 L/min/m². The feed flowrate should be adjusted by keeping TMP relatively constant at target pressure. The adjustment can be processed manually or automatically through Proportional-Integral-Derivative (PID) setting.

In some embodiments, the over concentrated pool in UF2 step can have concentration at 60 g/L, 180 g/L, 200 g/L, 240 g/L and any value from 50 g/L to 250 g/L at room temperature with solution viscosity up to 300 mPa·s. The UF2 pool made from over concentrated pool can have required concentration from 50 g/L to 243 g/L at room temperature by diluting with DF buffer. The protein concentration of UF2 pool for subcutaneous administration purpose requires high concentration, preferably higher than 150 g/L. In some embodiments, the UF2 pool has concentration at 167 g/L, 174 g/L, 184 g/L, 204 g/L and 243 g/L.

In some embodiments, the UF1 pool, DF pool, over concentrated pool and UF2 pool are stable at room temperature for 1 hour and up to 5 hours. The quality data (SEC-HPLC and CE-SDS(NR)) of protein are consistent during UF/DF process from UF1 to UF2 pool even in extremely high concentration 243 g/L processing and kept 5 hours at room temperature. In some embodiments, the final high concentrated drug substance manufactured by the UF/DF for subcutaneous administration according to the present disclosure has comparable quality data (SEC, CE-SDS(NR) and CZE) to the drug substance for intravenous infusion administration.

In some embodiments, the UF/DF unit operation is processed at 30° C. with buffers and all intermediate product pool kept at 30° C. The processing time at 30° C. can be ˜¼ less than time at room temperature. The over concentrated pool and UF2 pool are able to achieve up to 250 g/L at 30° C.

In some embodiments, the viscosity of protein solution is 1.56 mPa·s, 1.58 mPa·s in the feed, to about 33.47 mPa·s in UF1 pool and DF pool, up to 292.4 mPa·s in over concentrated pool and UF2 pool. The UF/DF process and system can handle solutions in a broad range of viscosity, up to 300 mPa·s.

In some embodiments, the formulation buffer is selected from histidine, acetate, mixture of histidine and acetic acid. In some embodiments, the formulation buffer can be histidine buffer. In some embodiments, the concentration of histidine buffer is from about 10 mM to about 30 mM. In some embodiments, the concentration of the histidine buffer is about 20 mM histidine.

TABLE 1

| | | | pH Shift During Freezing | |
|---|---|---|---|---|
| Acid for Buffer | Buffering Range | pKa | pH at 25° C. | Δ pH at −20° C. |
| Phosphoric acid | Neutral-Basic | 2.1, 7.2, 12.3 | 7.2 | −1.8 |
| Citric acid | Acidic-Neutral | 3.1, 4.8, 6.4 | 6.2 | −0.2 |
| Acetic acid | Acidic | 4.8 | 5.6 | +0.5 |
| Histidine | Neutral | 1.8, 6.1, 9.2 | 5.4 | +0.8 |
| Lactic acid | Acidic | 3.9 | N/A | N/A |
| Tromethamine | Neutral-Basic | 8.1 | 7.2 | +2.1 |
| Gluconic acid | Acidic | 3.6 | N/A | N/A |
| Aspartic acid | Acidic | 2.1, 3.9, 9.8 | N/A | N/A |
| Glutamic acid | Acidic | 2.1, 4.1, 9.5 | N/A | N/A |
| Tartaric acid | Acidic | 3.2, 4.9 | 5.0 | −0.3 |
| Succinic acid | Acidic-Neutral | 4.2, 5.6 | 5.6 | +0.3 |
| Malic acid | Acidic-Neutral | 3.4, 5.1 | 5.0 | −0.3 |
| Fumaric acid | Acidic | 3.0, 4.4 | N/A | N/A |
| α-Ketoglutaric | Acidic-Neutral | 2.5, 4.7 | N/A | N/A |

N/A—Data not available

In some embodiments the PD1 antibody is tislelizumab (BGB-A317, Table 2) or an antigen binding fragment of tislelizumab.

In some embodiments, the subcutaneous antibody formulation has an antibody concentration between about 50 mg to 800 mg. In another embodiment the subcutaneous antibody formulation has an antibody concentration of about 100 mg, about 150 mg, about 200 mg, about 250 mg, about 300 mg, about 350 mg, about 400 mg, about 450 mg, about 500 mg, about 550 mg or about 600 mg.

### BRIEF DESCRIPTION OF THE DRAWINGS

FIG. 1A. The process flowchart of high concentration UF/DF unit operation in detailed steps with UF1/DF/UF2 as main operation steps.

FIG. 1B. A diagram of UF/DF system designed for high concentration antibody solution processing.

FIG. 1C. Impacts of TMP and Feed Concentration on Permeate Flux.

FIG. 1D. Impacts of TMP and Feed flowrate on Permeate Flux.

FIG. 1E. UF1 intermediate pool protein concentration range with permeate flux*VCF.

FIG. 1F. The pH and conductivity curves in permeate flow change with diafiltration exchange volume for different UF1 intermediate pool concentration in Diafiltration step.

FIG. 1G. The impacts of TMP and feed flowrate on permeate flux with 50 g/L diafiltration pool solution in UF2 step.

FIG. 2A. The process chart of 167 g/L UF/DF unit operation with TMP, Feed flux, protein concentration and permeate flux curves in UF1, DF and UF2 steps.

FIG. 2B. The process chart of 167 g/L UF/DF unit operation with protein. concentration, osmolality and viscosity curves in UF1, DF and UF2 steps.

FIG. 3A. The quality data (SEC, CE-SDS(NR) and CZE) comparison between high concentration solution prepared by UF/DF in this example and current low concentration intravenous infusion solution.

FIG. 3B. The process chart of 174 g/L UF/DF unit operation with TMP, Feed flux, protein concentration and permeate flux curves in UF1, DF and UF2 steps.

FIG. 4A. The process chart of over-concentrated pool explore study with TMP, Feed flux, protein concentration and permeate flux curves in UF1, DF and UF2 steps.

FIG. 4B. The process chart of over-concentrated pool explore study with protein concentration, osmolality and viscosity curves in UF1, DF and UF2 steps.

FIG. 4C. SEC monomer data comparison of UF1 pool, DF pool and different over concentrated pool samples.

FIG. 4D. CE-SDS(NR) data comparison of UF1 pool, DF pool and different over-concentrated pool samples.

FIG. 4E. SEC monomer data comparison of UF1 pool, DF pool and maximum over-concentrated pool samples in 5-hours stability test at room temperature.

FIG. 4F. CE-SDS (NR) data comparison of UF1 pool, DF pool and maximum over-concentrated pool samples in 5-hours stability test at room temperature.

FIG. 5A. The process chart of 30° C. UF/DF process with TMP, Feed flux, protein concentration and permeate flux curves in UF1, DF and UF2 steps.

FIG. 5B. The pH and conductivity curves of 30° C. UF/DF process in DF step.

FIG. 5C. The process chart of 30° C. UF/DF process with protein concentration, osmolality and viscosity curves in UF1, DF and UF2 steps.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as ‘a,’ ‘an,’ and ‘the,’ include their corresponding plural references unless the context clearly dictates otherwise.

The term ‘or’ is used to mean, and is used interchangeably with, the term ‘and/or’ unless the context clearly dictates otherwise.

Throughout this specification and the claims which follow, unless the context requires otherwise, the word ‘comprise’, and variations such as ‘comprises’ and ‘comprising’, will be understood to imply the inclusion of a stated amino acid sequence, DNA sequence, step or group thereof, but not the exclusion of any other amino acid sequence, DNA sequence, step. When used herein the term ‘comprising’ can be substituted with the term ‘containing’, ‘including’ or sometimes ‘having’.

The term ‘antibody’ herein is used in the broadest sense and specifically covers antibodies (including full length monoclonal antibodies) and antibody fragments so long as they recognize antigen, e.g., PD1. An antibody is usually monospecific, but may also be described as idiospecific, heterospecific, or polyspecific. Antibody molecules bind by means of specific binding sites to specific antigenic determinants or epitopes on antigens.

The term ‘monoclonal antibody’ or ‘mAb’ or ‘Mab’ herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier ‘monoclonal’ indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256: 495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained by in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one ‘light chain’ (about 25 kDa) and one ‘heavy chain’ (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a ‘J’ region of about 12 or more amino acids, with the heavy chain also including a ‘D’ region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called ‘complementarity determining regions (CDRs)’, which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5th ed.; NIH Publ. No. 91-3242 (1991); Kabat (1978) Adv. Prot. Chem. 32: 1-75; Kabat, et al., (1977) J. Biol. Chem. 252: 6609-6616; Chothia, et al, (1987) J Mol. Biol. 196: 901-917 or Chothia, et al., (1989) Nature 342: 878-883.

The term ‘hypervariable region’ means the amino acid residues of an antibody that are responsible for antigen-binding. The hypervariable region comprises amino acid residues from a ‘CDR’ (i.e., VL-CDR1, VL-CDR2 and VL-CDR3 in the light chain variable domain and VH-CDR1, VH-CDR2 and VH-CDR3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term ‘framework’ or ‘FR’ residues mean those variable domain residues other than the hypervariable region residues defined herein as CDR residues.

Unless otherwise indicated, ‘antibody fragment’ or ‘antigen-binding fragment’ means antigen binding fragments of antibodies, i.e., antibody fragments that retain the ability to bind specifically to the antigen bound by the full-length antibody, e.g., fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered ‘specific’ for its intended target if its binding is determinative of the presence of the target protein in a sample, e.g., without producing undesired results such as false positives. Antibodies or binding fragments thereof, useful in the present disclosure will bind to the target protein with an affinity that is at least two-fold greater, preferably at least 10-times greater, more preferably at least 20-times greater, and most preferably at least 100-times greater than the affinity with non-target proteins. An antibody herein is said to bind specifically to a polypeptide comprising a given amino acid sequence.

The term ‘human antibody’ herein means an antibody that comprises human immunoglobulin protein sequences only. A human antibody may contain murine carbohydrate chains if produced in a mouse, in a mouse cell, or in a hybridoma derived from a mouse cell. Similarly, ‘mouse antibody’ or ‘rat antibody’ means an antibody that comprises only mouse or rat immunoglobulin protein sequences, respectively.

The term ‘humanized antibody’ means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix ‘hum,’ ‘hu,’ ‘Hu’ or ‘h’ is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

Further, the antibody of the present application has potential therapeutic uses in controlling viral infections and other human diseases that are mechanistically involved in immune tolerance or “exhaustion.’ In the context of the present application, the term ‘exhaustion’ refers to a process which leads to a depleted ability of immune cells to respond to a cancer or a chronic viral infection.

The term “trans-membrane pressure” or “‘TMP” is the pressure exerted on the UF/DF membrane. TMP is calculated by Equation (1) below:

$$TMP = \frac{{P}_{feed} + {P}_{retentate}}{2} - {P}_{permeate} \qquad Equation ( 1 )$$

in which, P_{feed}, Pᵣₑₜₑₙₜₐₜₑ, Pₚₑᵣₘₑₐₜₑ are pressure of feed inlet, retentate outlet and permeate outlet respectively.

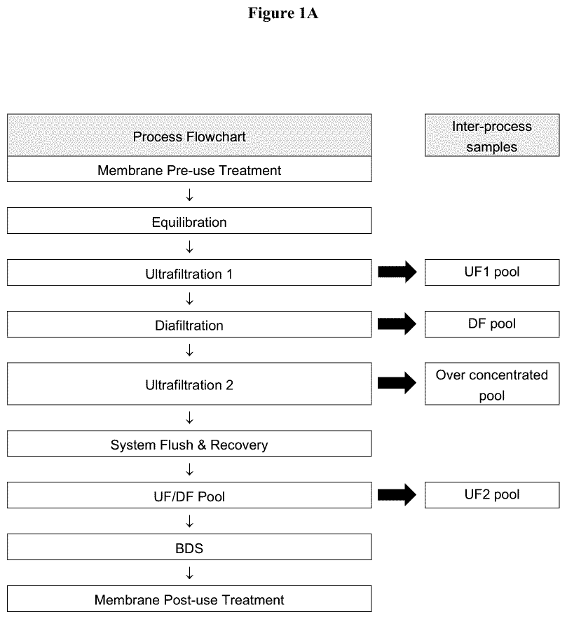

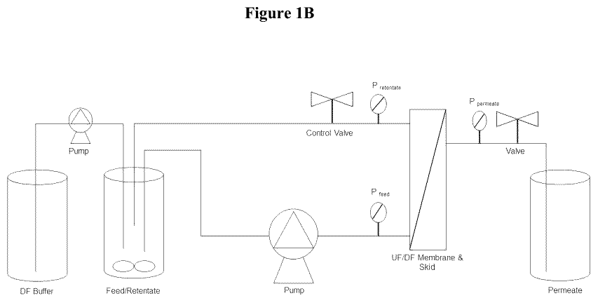

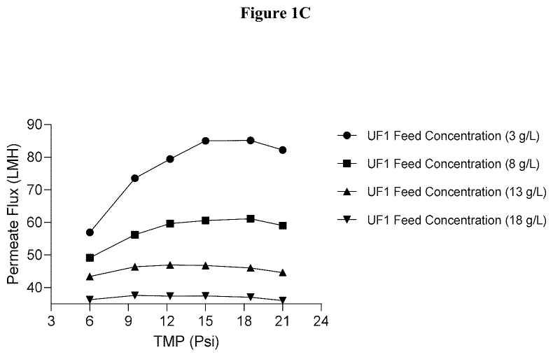

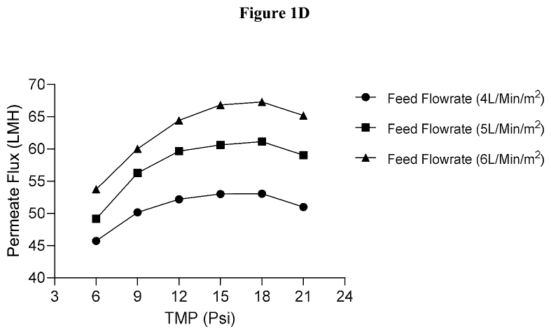

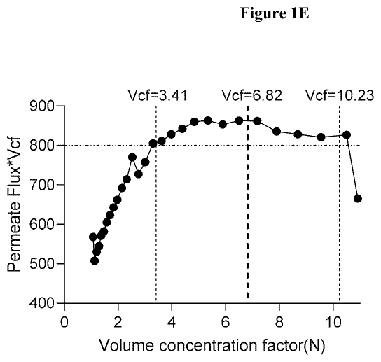

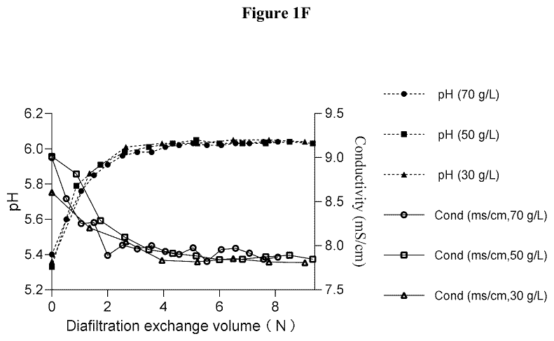

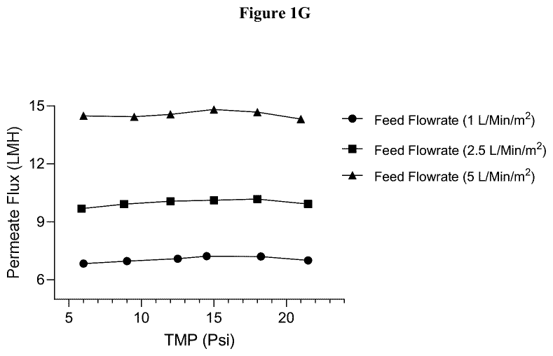

“Ultrafiltration step 1” (UF1) means the first ultrafiltration step in the process, this is shown in FIG. 1A.

“Diafiltration step” (DF) refers to any diafiltration step in the process, shown in FIG. 1A.

The term “ultrafiltration step 2” (UF2) means ultrafiltration step 2 in the process, shown in FIG. 1A.

The abbreviation “VCF” means “volume concentration factor,” which is the amount that the feed stream has been reduced in volume from the initial volume calculated by Equation (2):

$$Equation ( 2 )$$

$$VCF = \frac{Total initial feed volume}{Current retentate volume} = \frac{Current concentration in retentate}{Initial concentration in feed}$$

The term “WFI” means “water for injection.”

“CIP” means “clean-in-place.”

The term “NWP” is an abbreviation of “normalized water permeability”. The NWP test is a method to assess the effectiveness of the membrane CIP process.

The term “permeate flux” is defined as the solution flux through the UF/DF membrane.

### Anti-PD1 Antibody

The present disclosure provides for anti-PD1 antibodies and subcutaneous formulations thereof. For example, Tislelizumab (BGB-A317), is an anti-PD1 antibody disclosed in U.S. Pat. No. 8,735,553 with the sequences provided below.

TABLE 2
Tislelizumab sequences

| | SEQ | |
|---|---|---|
| | ID | |
| Domain | NO: | Amino Acid Sequence |
| HCDR1 | SEQ | GFSLTSYGVH |
| | ID | |
| | NO: | |
| | 1 | |
| HCDR1 | SEQ | VIYADGSTNYNPSLKS |
| | ID | |
| | NO: | |
| | 2 | |
| HCDR3 | SEQ | ARAYGNYWYIDV |
| | ID | |
| | NO: | |
| | 3 | |
| LCDR1 | SEQ | KSSESVSNDVA |
| | ID | |
| | NO: | |
| | 4 | |
| LCDR2 | SEQ | YAFHRFT |
| | ID | |
| | NO: | |
| | 5 | |
| LCDR3 | SEQ | HQAYSSPYT |
| | ID | |
| | NO: | |
| | 6 | |
| VH | SEQ | QVQLQESGPGLVKPSETLSLTCTVSGFSLT |
| | ID | SYGVHWIRQPPGKGLEWIGVIYADGSTNYN |
| | NO: | PSLKSRVTISKDTSKNQVSLKLSSVTAADT |
| | 7 | AVYYCARAYGNYWYIDVWGQGTTVTVSS |
| VL | SEQ | DIVMTQSPDSLAVSLGERATINCKSSESVS |
| | ID | NDVAWYQQKPGQPPKLLINYAFHRFTGVPD |
| | NO: | RFSGSGYGTDFTLTISSLQAEDVAVYYCHQ |
| | 8 | AYSSPYTFGQGTKLEIK |
| IgG4 | SEQ | ASTKGPSVFPLAPCSRSTSESTAALGCLVK |
| constant | ID | DYFPEPVTVSWNSGALTSGVHTFPAVLQSS |
| domain | NO: | GLYSLSSVVTVPSSSLGTKTYTCNVDHKPS |
| | 9 | NTKVDKRVESKYGPPCPPCPAPPVAGGPSV |
| | | FLFPPKPKDTLMISRTPEVTCVVVAVSQED |
| | | PEVQFNWYVDGVEVHNAKTKPREEQFNSTY |
| | | RVVSVLTVVHQDWLNGKEYKCKVSNKGLPS |
| | | SIEKTISKAKGQPREPQVYTLPPSQEEMTK |
| | | NQVSLTCLVKGFYPSDIAVEWESNGQPENN |
| | | YKTTPPVLDSDGSFFLYSKLTVDKSRWQEG |
| | | NVFSCSVMHEALHNHYTQKSLSLSLGK |
| Heavy | SEQ | QVQLQESGPGLVKPSETLSLTCTVSGFSLT |
| Chain | ID | SYGVHWIRQPPGKGLEWIGVIYADGSTNYN |
| | NO: | PSLKSRVTISKDTSKNQVSLKLSSVTAADT |
| | 10 | AVYYCARAYGNYWYIDVWGQGTTVTVSSAS |
| | | TKGPSVFPLAPCSRSTSESTAALGCLVKDY |
| | | FPEPVTVSWNSGALTSGVHTFPAVLQSSGL |
| | | YSLSSVVTVPSSSLGTKTYTCNVDHKPSNT |
| | | KVDKRVESKYGPPCPPCPAPPVAGGPSVFL |
| | | FPPKPKDTLMISRTPEVTCVVVAVSQEDPE |
| | | VQFNWYVDGVEVHNAKTKPREEQFNSTYRV |
| | | VSVLTVVHQDWLNGKEYKCKVSNKGLPSSI |
| | | EKTISKAKGQPREPQVYTLPPSQEEMTKNQ |
| | | VSLTCLVKGFYPSDIAVEWESNGQPENNYK |
| | | TTPPVLDSDGSFFLYSKLTVDKSRWQEGNV |
| | | FSCSVMHEALHNHYTQKSLSLSLGK |
| Light chain | SEQ | DIVMTQSPDSLAVSLGERATINCKSSESVS |
| | ID | NDVAWYQQKPGQPPKLLINYAFHRFTGVPD |
| | NO: | RFSGSGYGTDFTLTISSLQAEDVAVYYCHQ |
| | 11 | AYSSPYTFGQGTKLEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |

Anti-PD1 antibodies can include, without limitation, Tislelizumab, Pembrolizumab or Nivolumab. Pembrolizumab (formerly MK-3475), as disclosed by Merck, in U.S. Pat. Nos. 8,354,509 and 8,900,587 is a humanized IgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human IgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Antibody Production

Anti-PD1 antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide that encodes for the polypeptide of SEQ ID NO:7. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide that encodes for the polypeptide of SEQ ID NO:8.

The polynucleotides of the present disclosure can encode the variable region sequence of an anti-PD1 antibody. They can also encode both a variable region and a constant region of the antibody. Some of the polynucleotide sequences encode a polypeptide that comprises variable regions of both the heavy chain and the light chain of one of the exemplified Tislelizumab antibodies.

Also provided in the present disclosure are expression vectors and host cells for producing the Tislelizumab antibodies. The choice of expression vector depends on the intended host cells in which the vector is to be expressed. Typically, the expression vectors contain a promoter and other regulatory sequences (e.g., enhancers) that are operably linked to the polynucleotides encoding a Tislelizumab antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of a Tislelizumab antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20: 125, 1994; and Bittner et al., Meth. Enzymol., 153: 516, 1987). For example, the SV40 enhancer or CMV enhancer can be used to increase expression in mammalian host cells.

The host cells for harboring and expressing the Tislelizumab antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express Tislelizumab. Insect cells in combination with baculovirus vectors can also be used.

In other aspects, mammalian host cells are used to express and produce Tislelizumab. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89: 49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### EXAMPLES

The examples and description of certain embodiments should be taken as illustrating, rather than as limiting the present disclosure as defined by the claims. As will be readily appreciated, numerous variations and combinations of the features set forth above can be utilized without departing from the present disclosure and as set forth in the claims. All such variations are intended to be included within the scope of the present disclosure. All references cited are incorporated herein by reference in their entireties.

### Analytical Methods

This methods section provides a summary of the methods used in the following Examples 1-5.

### SEC-HPLC

Formation of soluble aggregates is analyzed by size exclusion chromatography (SEC) on a Waters HPLC system. Protein is separated based on molecular size on a TSKgel G3000™ SWXL column maintained at 37±5 C° using an isocratic gradient. Molecular weight species are eluted and detected by UV absorption at 280 nm. The distribution of aggregates, monomer and fragments are quantitated via the peak areas for standards and samples.

### CZE

The charge heterogeneity of a sample is determined using PA800 Plus™ (Beckman) by a capillary zone electrophoresis method (CZE) also known as free solution capillary electrophoresis. Samples are separated based on their electrophoretic mobilities caused by differences in charge and hydrodynamic radius of the analytes in a capillary filled with a buffer solution containing caproic acid. The samples are analyzed in their native state when an external electric field is applied resulting in a specific peak pattern showing the various charge variants of the antibody (acidic, basic and main charge variants). Samples are injected by pressure and the mobilized proteins are detected by UV absorbance at 214 nm.

### CE-SDS(NR)

The purity of sample is determined using PA800 Plus™ (Beckman) by a capillary gel electrophoresis (CE) method. Samples are denatured with sodium dodecyl sulphate (SDS) and separated based on size in a capillary filled with a gel that acts as a sieving medium. In non-reduced (NR) samples, an alkylating agent, N-Ethylmaleimide (NEM), is added to avoid any fragmentation induced by sample preparation and to ensure that the main IgG peak remains intact. Samples are injected electrokinetically and the mobilized proteins are detected by UV absorbance at 200 nm using a UV detector. The reportable value for non-reduced samples is the time corrected area percent (TCA) % of the IgG main peak.

### Protein Concentration

Protein concentrations are determined at UV 280 nm.

### Viscosity

The viscosity of the antibody formulations is measured on a chip-based micro VISC™ instrument (Rheosense), in which the pressure difference correlates with solution dynamic viscosity. Sample size is approximately 70-100 μL. Aliquots are loaded into a 400 μL micro VISC™ disposable pipette and connected to the chip. Triplicate measurements are taken at a shear rate of 500 S⁻¹ and at a temperature of about 25° C.

### Osmolality

The osmolality of the antibody solution or buffer solution is measured by OSMOMAT 3000™ osmolality tester (Gonotec). 50 μl of each sample is loaded twice and tested to obtain the average osmolality value.

### Example 1: Defining Parameters of the UF1/DF/UF2 Process for High Concentration PD1 Antibody Solution

In order to define the parameters of the UF/DF for a high concentration PD1 antibody solution, a lab scale UF/DF system and process were designed to implement scale up and future large scale GMP production. The unit operation contained several steps: membrane pre-use treatment (WFI flush, Integrity test, CIP, NWP test), equilibrium, ultrafiltration 1, diafiltration, ultrafiltration 2, system flush and recovery, UF/DF pool, membrane post-use treatment etc., which are shown in the process flowchart (FIG. 1A). The UF/DF system was comprised of a selected UF/DF membrane and membrane housing skid, three pressure gauges for feed, retentate and permeate fluid flow path, valves at retentate and permeate outlet for TMP and flowrate adjusting, one pump for feed loading, one pump for buffer supplement, and three containers for feed/retentate, buffer and permeate solutions, shown in the diagram in FIG. 1B. The fluid paths in this system were well designed to minimize the system dead volume in order to reduce the dilution effect by system flush.

In the first step to determine the process parameters for ultrafiltration 1 (UF1), Tislelizumab was prepared and purified after a viral filtration step as the UF/DF process feed solution. The antibody was dispersed in a process feed solution of 50 mM acetate, pH 5.36 buffer with an antibody concentration of 3, 8, 13 or 18 g/L and filtered by 0.2 μm Corning™ filtration system. To evaluate the TMP-Flux relationship, a Pellion3 Ultracel™ 30 kDa, D membrane with 0.11 m² area was used in the lab scale UF/DF process design and testing. The recommended feed flowrate of the Pellicon3™ membrane in this example was 4-6 L/min/m². The permeate flux was monitored during all processing conditions.

FIG. 1C shows the impacts of TMP and feed concentration on permeate flux under feed flowrate 5 L/min/m² in the UF1 step. With the TMP increasing, permeate flux also increased accordingly. As the process continued, a high concentration antibody protein layer formed on the surface of membrane and permeate flux achieved the optimal point. Further concentrated, it caused flux decreasing. In a 3 g/L feed solution, the optimal TMP was about 15-21 Psi. For higher feed concentration at 13 or 18 g/L, the optimal TMP was approximately 6-18 Psi due to the high concentration layer that formed much earlier than 3 g/L feed condition. The TMP was controlled at 6-21 Psi for a different feed for UF1 step.

FIG. 1D shows the impacts of TMP and feed flowrate on permeate flux with 8 g/L feed solution in the UF1 step. With different feed flowrate at 4, 5 and 6 L/min/m², the optimal permeate flux can be all achieved by controlling TMP at 15-18 Psi. The lower TMP controlled at 9-15 Psi, did not decrease permeate flux significantly, which is acceptable in the high concentration UF/DF process. The feed flowrate at 4-6 L/min/m² was the best operating range for the UF1 step.

FIG. 1E shows the UF1 intermediate pool protein concentration range during the UF1 step with the value of permeate flux*VCF. The initial antibody protein concentration in the feed was 7.33 g/L. The higher value of permeate flux*VCF, the better ultrafiltration effect in UF1 step and diafiltration effect in the later DF step. Controlled TMP at 15 Psi and feed flowrate 5 L/min/m², the value of permeate flux*VCF kept near maximum in the antibody protein range of 25 g/L to 75 g/L, and had a quick drop at about 80 g/L due to the Donnan effect. The Donnan effect, also known as the Gibbs-Donnan effect or Donnan's Law is a description of the behavior of charged particles (such as proteins) that fail to distribute evenly across the two sides of the membrane. The broad UF1 intermediate pool protein concentration range: 25 g/L to 75 g/L, provided process robustness in the following DF and UF2 steps. The corresponding VCF was from 3.41 (25 g/L/7.33 g/L) to 10.23 (75 g/L/7.33 g/L), and preferably not more than 25 (75 g/L/3 g/L). The concentration ratio in the UF2 step can be 2-3 to achieve a high antibody concentration solution at 50 g/L to 75 g/L from low end 25 g/L UF1 intermediate pool. The concentration ratio can be as low as 3.2 (˜240 g/L/75 g/L) to achieve extremely high concentration solution, even at ˜240 g/L in the UF2 step from the initial 75 g/L UF1 intermediate pool.

FIG. 1F shows the pH and conductivity curves in the permeate flow change with diafiltration exchange volume for different UF1 intermediate pool concentrations in the diafiltration (DF) step. Controlled diafiltration TMP at 15 Psi and feed flowrate 5 L/min/m², the pH and conductivity curves became flat and values were same as DF buffer after 4 exchange volume (Table 2), which indicated the DF step could be considered completed if the volume exchange number was larger than 4.

TABLE 2
The pH and conductivity of protein solution from different
initial UF1 pool concentrations after about 4 exchange
volumes show same values as DF buffer

| UF1 pool | Exchange volume | pH | Conductivity (mS/cm) |
|---|---|---|---|
| 70 g/L | 4.01 | 6.01 | 7.935 |
| 50 g/L | 4.32 | 6.03 | 7.913 |
| 30 g/L | 3.94 | 6.03 | 7.835 |
| DF buffer | N/A | 6.05 | 7.866 |

The impact of TMP and feed flowrate on permeate flux is important to monitor with the 50 g/L diafiltration pool solution in the UF2 step. The permeate flux curves were kept flat at a TMP range from 6 to 22 Psi in all feed flowrate conditions. It demonstrated that the initial diafiltration pool concentration 50 g/L already formed high concentration protein layer on the membrane surface and there was no optimal TMP for permeate flux in UF2 step (FIG. 1G). The permeate flux was only proportional to feed flowrate in the UF2 step. During UF2, the solution concentration and viscosity increased with process progressing, which further increased TMP. Thus, the TMP can be controlled at certain target value, for example, 15 Psi, or a range, such as 6 to 29 Psi in UF2, by adjusting the feed flowrate from initial target flowrate, for example, 5 L/min/m² to a lower value, 1 L/min/m².

### Example 2: UF/DF Unit Operation for 167 g/L Protein Pool Manufacturing

Tislelizumab was prepared and purified after viral filtration as UF/DF process feed solution as described in Example 1. The antibody was dispersed in 50 mM acetate, pH 5.37 buffer with concentration at 7.97 g/L. Membrane A: 0.11 m² area Pellion3 Ultracel™ 30 kDa, D membrane, with the lab scale UF/DF system was used for the UF/DF process. The loading capacity for Membrane A was 229.0 g/m². The unit operation contained membrane pre-use treatment (WFI flush, Integrity test, CIP, NWP test), equilibrium, ultrafiltration 1, diafiltration, ultrafiltration 2, system flush and recovery, UF/DF pool, membrane post-use treatment etc., as shown in the process flowchart (FIG. 1A).

The TMP was controlled at about 14.5 Psi and feed flowrate at 218 LMH in UF1 step. The antibody protein concentration was concentrated to 48.5 g/L, with a viscosity of 1.58 mP·s. The same TMP and feed flowrate were controlled in the following DF step. After 6 exchanges of DF buffer (20 mM His-His HCl, 70 mM NaCl with pH 6.04), the DF pool solution has an antibody protein concentration at 46.73 g/L with pH at 5.99. The DF pool solution was further processed in UF2 step with TMP controlled at about 14.5 Psi by adjusting feed flowrate lower to 109 LMH gradually with protein concentration increasing. The over concentrated pool achieved 191 g/L protein concentration with viscosity at 33.47 mPa·s. After flushing and recycling the whole UF/DF system with a volume of DF buffer, the final UF2 pool had an antibody protein concentration at 167 g/L, in 20 mM His-His HCl, 70 mM NaCl, pH 6.1 buffer.

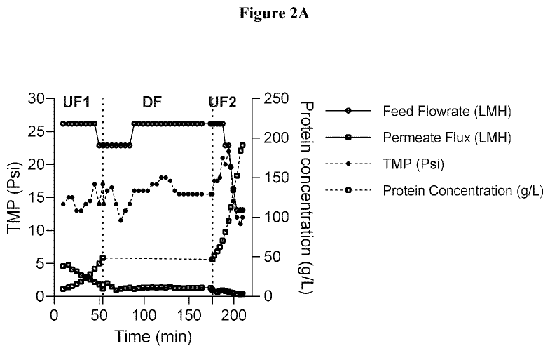

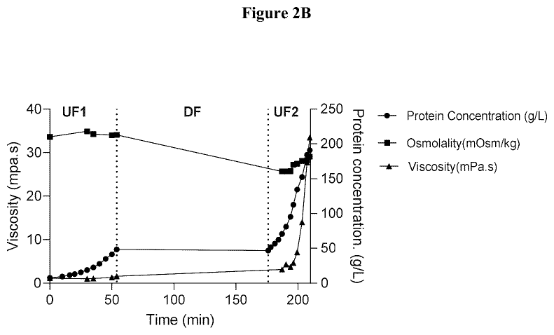

FIG. 2A shows the process chart of 167 g/L UF/DF unit operation. Protein concentration increased in both UF1 and UF2 steps. In UF2 step, antibody protein concentration increased significantly to a high range (>100 g/L), which required a manual decrease in the feed flowrate by keeping TMP at the target value 14.5 Psi. FIG. 2B shows the osmolality and viscosity curves with antibody protein concentration changing in UF1/DF/UF2 steps. Osmolality and viscosity increased exponentially when antibody protein concentration was beyond 100 g/L in the UF2 step. This was consistent with the permeate flux decreasing phenomenon observed in FIG. 2A, at the UF2 step. This example also shows the UF/DF system and currently designed processes were suitable to manufacture the final antibody protein solution with viscosity about 33.47 mPa·s.

### Example 3: UF/DF Unit Operation for 174 g/L Protein Pool Manufacturing

Tislelizumab was prepared and purified after viral filtration as UF/DF process feed solution as described in Example 1. The antibody was dispersed in 50 mM acetate, pH 5.27 buffer with concentration of 8.27 g/L. Membrane B: three 0.14 m² area Sartocon Slice ECO Hydrosart™ 30 kDa membranes (total area 0.42 m²), with the lab scale UF/DF system was used in the UF/DF process. The loading capacity for Membrane B was 739.7 g/m². The unit operation contained membrane pre-use treatment (WFI flush, Integrity test, CIP, NWP test), equilibrium, ultrafiltration 1, diafiltration, ultrafiltration 2, system flush and recovery, UF/DF pool, membrane post-use treatment etc., are shown in the process flowchart (FIG. 1A).

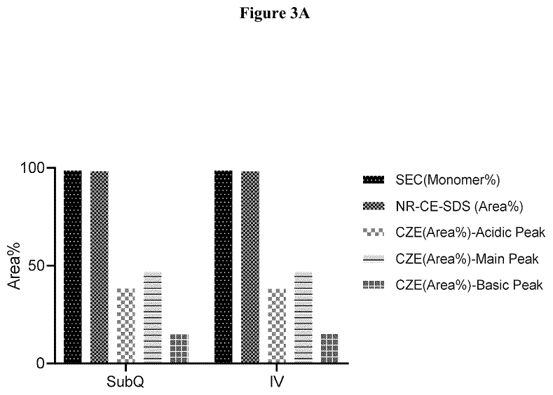

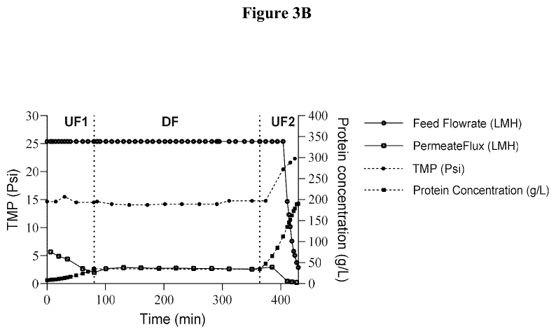

The TMP was controlled at about 14.5 Psi and feed flowrate at 338.57 LMH in UF1 step. The protein concentration was concentrated to 34.47 g/L in UF1 pool. The same TMP and feed flowrate were controlled in the following DF step. After 8 exchange volume of DF buffer (20 mM His-His HCl, 70 mM NaCl with pH 6.01), the DF pool solution has protein concentration at 34.64 g/L with pH at 5.98. The DF pool solution was further processed in UF2 step with TMP controlled at about 14.5 Psi (no more than 29 Psi) by adjusting feed flowrate lower to 38.57 LHM gradually with antibody protein concentration increasing. The over concentrated pool achieved 190.34 g/L antibody protein concentration. After flushing and recycling the whole UF/DF system with a volume of DF buffer, the final UF2 pool had an antibody protein concentration at 173.98 g/L, in 20 mM His-His HCl, 70 mM NaCl, pH 6.0 buffer. The quality data (SEC, CE-SDS(NR) and CZE) shown in FIG. 3A demonstrated the final high protein concentration solution (174 g/L) prepared by this UF/DF process were comparable to current 10 g/L intravenous infusion solution, indicating that the concentration process maintained the integrity of the Tislelizumab antibody.

FIG. 3B shows the process chart of 174 g/L UF/DF unit operation. Antibody protein concentration increased in both the UF1 and UF2 steps. Due to larger membrane area and the UF/DF skid (as shown in FIG. 1B), the TMP and feed flowrate were steadily controlled at 14.5 Psi and 338.57 LMH during UF1 and DF steps. In UF2, TMP increased with protein concentration increasing. Thus, TMP was controlled by lowering feed flowrate to about 38.57 LMH.

### Example 4: Evaluation of the Maximum Antibody Protein Concentration in the Final UF/DF Step and Antibody Stability

In order to explore the antibody protein concentration range and stability of the concentrated antibody solution in UF2 step, a new set of UF/DF experiments were designed by continuing the UF2 step until the solution viscosity achieves about 300 mPa·s. Tislelizumab was prepared and purified after viral filtration as UF/DF process feed solution as described above. The antibody was dispersed in 50 mM acetate, pH 5.36 buffer with concentration at 8.15 g/L. Membrane A: 0.11 m² area Pellion3 Ultracel™ 30 kDa, D membrane, with the lab scale UF/DF system was used for UF/DF processing. The loading capacity for Membrane A was 601.67 g/m². The unit operation contained membrane pre-use treatment (WFI flush, Integrity test, CIP, NWP test), equilibrium, ultrafiltration 1, diafiltration, ultrafiltration 2, system flush and recovery, UF/DF pool, membrane post-use treatment etc., as shown in the process flowchart (FIG. 1A). The solution was firstly concentrated to 50 g/L in UF1 with viscosity 1.56 mPa·s. Then the solution was diafiltrated with 6 exchange volumes of DF buffer (20 mM His-His HCl, 70 mM NaCl with pH 6.04) to obtain the initial testing material for UF2.

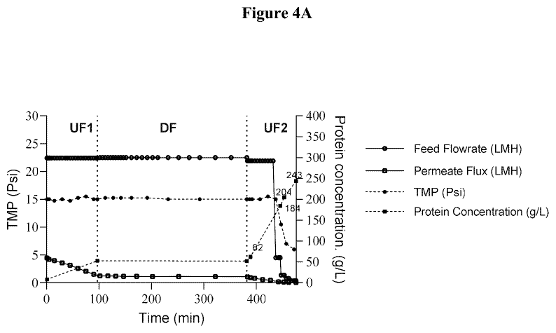

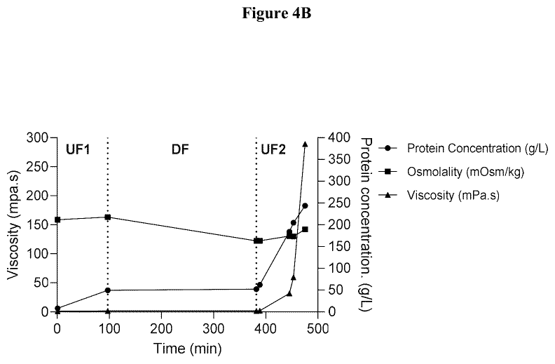

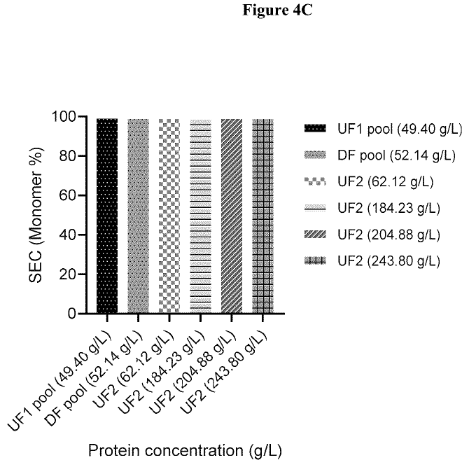

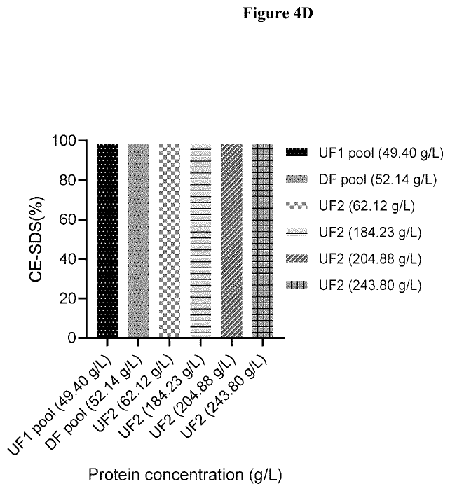

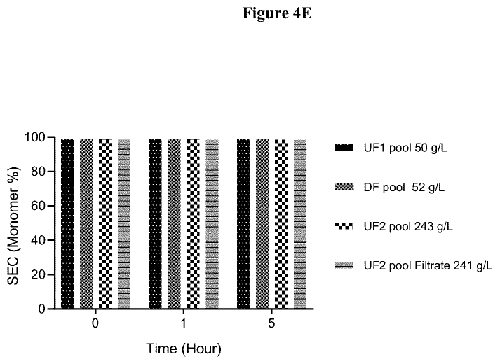

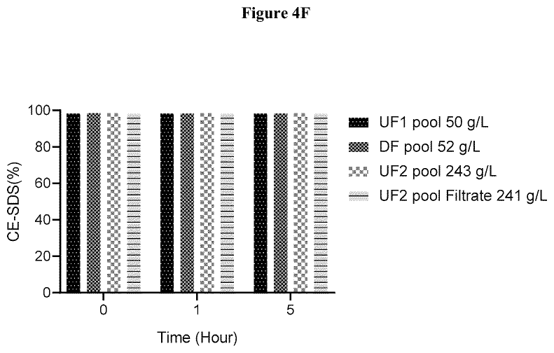

FIG. 4A shows the process chart of concentrated antibody until the antibody achieved a concentration of 243 g/L. TMP and feed flowrate was kept constant and well controlled at 15±0.5 Psi and 300 LMH in the UF1 and DF steps. In the UF2 step, with protein concentration increasing, osmolality and viscosity increased significantly, as demonstrated in FIG. 4B. TMP and feed flowrate needed adjusting simultaneously when protein concentration was beyond 150 g/L, in order to keep TMP less than 29 Psi but still have continuous permeate flux through the membrane. During UF2, three inter-process samples were taken at protein concentration at 62, 184 and 204 g/L for quality analysis and stability tests. When the concentration approached the final 243 g/L concentration, viscosity was about 300 mPa·s and thus caused both feed flowrate and permeate flux close to zero, which indicated that the limits of the UF2 step had been reached.

FIG. 4C and FIG. 4D show the quality attributes (SEC and CE-SDS(NR)) of UF1 pool, DF pool, and four concentrated antibody pool samples from the above process at room temperature. There were no quality differences between these samples, even in the 243 g/L sample. FIG. 4E and FIG. 4F show the quality attributes (SEC and CE-SDS(NR)) comparison of a UF1 pool, a DF pool and a maximum concentrated antibody pool sample during a 5 hour stability test. The results show all inter-process samples were stable for 5 hours, which demonstrated the robustness of this UF/DF process even for extremely high concentration antibody processing. As the over concentrated pool can achieve 243 g/L, with different system flush strategy and trade off with yield, the protein concentration range of final UF2 pool can flexibly cover 50 g/L (low end of UF1 pool 25 g/L with 2-time concentrated factor in UF2) to 243 g/L (maximum concentrated pool without system flush by accepting relatively lower yield).

### Example 5: UF/DF Unit Operation for Extremely High Protein Concentration at Higher Operation Temperature

It is known that solution viscosity decreases with solution temperature increasing. Theoretically, operating UF/DF at higher temperate, for example, 30° C., will show better process performance, like more even TMP and better flux control, or be able to achieve higher concentration than the process at room temperature or lower temperature. To evaluate the temperature effects on this UF/DF process, Tislelizumab was prepared and purified after viral filtration as UF/DF process feed solution as described above in Example 1. The antibody was dispersed in 50 mM acetate pH 5.27 buffer with concentration at 8.08 g/L. Membrane A: 0.11 m² area Pellion3 Ultracel™ 30 kDa, D membrane, with the lab scale UF/DF system was used for UF/DF processing. The loading capacity for Membrane A was 585.2 g/m². The unit operation contained membrane pre-use treatment (WFI flush, Integrity test, CIP, NWP test), equilibrium, ultrafiltration 1, diafiltration, ultrafiltration 2, system flush and recovery, UF/DF pool, membrane post-use treatment etc., as shown in the process flowchart (FIG. 1A). The feed solution and buffer were stored in individual containers and kept at 30° C. controlled by a water bath.

The TMP was controlled at about 15 Psi and feed flowrate at 300 LMH in the UF1 step. The antibody was concentrated to 48.77 g/L, with a viscosity of 1.56 mPa·s. The same TMP and feed flowrate were controlled in the following DF step. After 6 exchange volumes of DF buffer (20 mM His-His HCl, 70 mM NaCl with pH 5.99), the DF pool solution has an antibody protein concentration of 49.15 g/L with pH at 5.99. The DF pool solution was further processed in the UF2 step with TMP controlled at about 15 Psi (no more than 29 Psi) by adjusting feed flowrate lower to 120 LMH gradually as antibody protein concentration increased. The concentrated antibody pool achieved a protein concentration of 248.54 g/L with viscosity 292.4 mPa·s in 20 mM His-His HCl, 70 mM NaCl, pH 6.06 buffer.

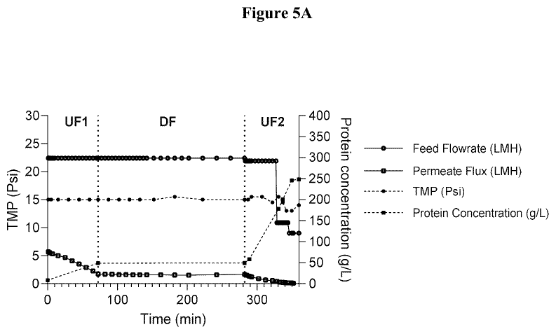

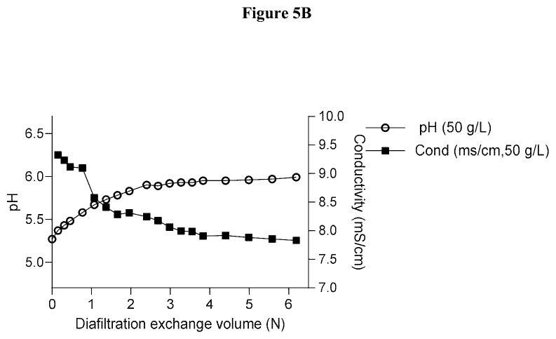

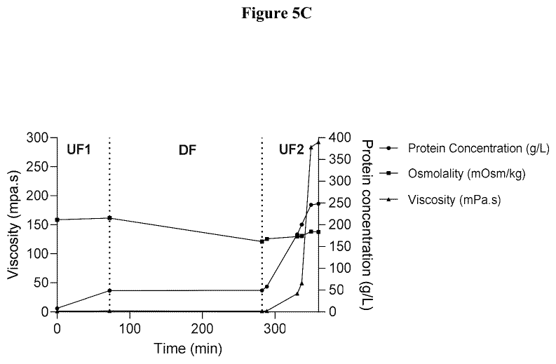

FIGS. 5A, 5B, 5C show the process chart of TMP, Feed flux, antibody protein concentration and permeate flux curves in UF1, DF and UF2 steps; pH and conductivity curves in DF step; protein concentration, osmolality and viscosity curves in UF1, DF and UF2 steps, respectively. The trends of each curve in all steps were similar to those in Example 2, Example 3 and Example 4. Due to lower viscosity at 30° C., permeate flux was slightly higher than process operated at room temperature in previous examples under the same TMP. The permeate flux was 22.9 LMH at 30° C. and 16.8 LMH at room temperature in the UF1/DF step. The processing time in UF1 at 30° C. was 72.2 min versus 97 min at room temperature. The time for target 6 exchange volumes during the DF step at 30° C. was also much less: 210 min (30° C.) versus 285 min (RT).

Reflected to the process chart, the total operation time to achieve required target protein concentration was shorter at 30° C., especially in the UF2 step. For the 200 g/L target antibody concentration in the UF2 step, it only took 54.79 minutes at 30° C. with a mean permeate flux 10.06 LMH. In contrast, lowering the temperature to room temperature required 71.8 minutes with a mean permeate flux 7.03 LMH. Also, relatively higher concentration can be achieved. Therefore, if extremely high concentration protein solution, for example, up to 250 g/L, is necessary, it can be manufactured by the process shown in this example, with the operation temperature controlled at 30° C.

1. An ultrafiltration (UF)/diafiltration (DF) process for a highly concentrated solution comprising a PD1 antibody or antigen binding fragment thereof, the process comprising the steps of:
A. ultrafiltrating the PD1 antibody or antigen binding fragment thereof in a process feed material (ultrafiltration 1 (UF1)) to obtain a UF1 pool protein with an intermediate antibody concentration;
B. diafiltrating the UF1 pool protein from step A with diafiltration (DF) buffer into a final drug substance formulation buffer, to obtain a DF pool;
C. ultrafiltrating the DF pool from step B into a high concentration solution as over concentrated pool with a desired concentration; and
D. adjusting the over concentrated pool to a final drug substance target concentration to prepare a UF2 pool, and then further diluting the UF2 pool to the concentration solution.

2. The process of claim 1, wherein the PD1 antibody or antigen binding fragment thereof, comprises;
(a) a HCDR (Heavy Chain Complementarity Determining Region) 1 of SEQ ID NO: 1.
(b) a HCDR2 of SEQ ID NO:2, and
(c) a HCDR3 of SEQ ID NO:3 and a light chain variable region that comprises:
(i) a LCDR (Light Chain Complementarity Determining Region) 1 of SEQ ID NO: 4,
(ii) a LCDR2 of SEQ ID NO:5, and
(iii) a LCDR3 of SEQ ID NO:6.

3. The process of claim 1, wherein the PD1 antibody or antigen binding fragment thereof, comprises SEQ ID NO:7 and SEQ ID NO:8.

4. The process of claim 1, wherein the feed material in step A comprises a buffer, wherein the buffer is histidine, acetate, citrate, succinate, phosphate, a mixture of histidine and acetic acid, or a mixture of histidine and citric acid.

5. The process of claim 4, wherein the feed material in step A comprises a buffer, wherein the buffer is histidine, a mixture of histidine and acetic acid or a mixture of histidine and citric acid.

6. The process of claim 1, wherein the highly concentrated solution is at a concentration of 3 g/L to 18 g/L.

7. The process of claim 1, wherein the steps A-C comprise a 30 kDa or a 50 kDa membrane.

8. The process of claim 7, wherein the membrane loading capacity is 100 g/m² to 800 g/m².

9-11. (canceled)

12. The process of claim 1, wherein in step A the UF1 pool protein concentration is a range of 25-75 g/L.

13. The process of claim 1, wherein step A results in a volume concentration factor (VCF) in the range of 2 to 25.

14-16. (canceled)

17. The process of claim 1, wherein in step B, the UF1 pool protein has a protein concentration between 25-75 g/L.

18. The process of claim 17, wherein the UF1 pool protein has a concentration of about 50 g/L.

19-25. (canceled)

26. The process of claim 1, wherein in step C the DF pool has a protein concentration between 25-75 g/L.

27. The process of claim 26, wherein the protein concentration is about 50 g/L.

28. The process of claim 1, wherein in step D the over concentrated pool has a protein concentration from 60 g/L to 250 g/L.

29. The process of claim 1, wherein the UF2 pool in step D is prepared by diluting the over concentrated pool to a concentration of 60 g/L to 250 g/L.

30. The process of claim 29, wherein the UF2 pool is prepared by diluting the overconcentrated pool to 167 g/L.

31. The process of claim 30, wherein the UF2 pool in step D is buffered with histidine.

32. The process of claim 31, wherein the concentration of histidine is 15 mM to 25 mM.

33. The process of claim 32, wherein the buffer comprises 20 mM histidine buffer with pH between 5.5-6.0.

35. (canceled)


SEQUENCE LISTING (11 sequences)

SEQ ID NO: 1 - protein, 10 aa, synthetic construct
  REGION 1..10: note = HCDR1
GFSLTSYGVH

SEQ ID NO: 2 - protein, 16 aa, synthetic construct
  REGION 1..16: note = HCDR1
VIYADGSTNYNPSLKS

SEQ ID NO: 3 - protein, 12 aa, synthetic construct
  REGION 1..12: note = HCDR3
ARAYGNYWYIDV

SEQ ID NO: 4 - protein, 11 aa, synthetic construct
  REGION 1..11: note = LCDR1
KSSESVSNDVA

SEQ ID NO: 5 - protein, 7 aa, synthetic construct
  REGION 1..7: note = LCDR2
YAFHRFT

SEQ ID NO: 6 - protein, 9 aa, synthetic construct
  REGION 1..9: note = LCDR3
HQAYSSPYT

SEQ ID NO: 7 - protein, 118 aa, synthetic construct
  REGION 1..118: note = VH
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 8 - protein, 107 aa, synthetic construct
  REGION 1..107: note = VL
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 9 - protein, 327 aa, synthetic construct
  REGION 1..327: note = IgG4 constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 10 - protein, 445 aa, synthetic construct
  REGION 1..445: note = Heavy Chain
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 11 - protein, 214 aa, synthetic construct
  REGION 1..214: note = Light chain
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC